(19) United States
(12) Patent Application Publication
(10) Pub. No.: US 2018/0082034 A1
(43) Pub. Date: Mar. 22, 2018

(54) SURGICAL KIT TRACKING CONTROL DEVICE

(71) Applicant: HCL Technologies Limited, Noida (IN)

(72) Inventors: Lalit Kumar Singh, Noida (IN); Pankaj GUPTA, Noida (IN)

(21) Appl. No.: 15/666,785

(22) Filed: Aug. 2, 2017

(30) Foreign Application Priority Data
Sep. 21, 2016 (IN) 201611032247

(51) Int. Cl. G06F 19/00; A61B 34/20; A61B 50/31

(52) U.S. Cl. CPC G06F 19/3406; G06F 19/3418; A61B 34/20; A61B 90/98; A61B 2034/2048; A61B 2034/2051; A61B 50/31

(57) ABSTRACT

Disclosed is a device and method for controlling operations of a tracking device installed in a surgical kit. The method includes receiving one or more signals from an accelerometer in which the accelerometer may be configured to transmit the one or more signals upon detecting change in a state of the surgical kit. Further, the state may be a stationary state and/or an in-motion state. The method further includes activating the tracking device based on the one or more signals and obtaining a location co-ordinate of the surgical kit from the tracking system. The method furthermore includes transmitting the state of the surgical kit and the location co-ordinate of the surgical kit via a communication channel based on a signal strength of the communication channel and deactivating the tracking device, thereby controlling operations of a tracking device installed in a surgical kit.

### CROSS-REFERENCE TO RELATED APPLICATIONS AND PRIORITY

The present application claims priority from the Indian Patent Application no. 201611032247 filed on Sep. 21, 2016, the entirety of which is incorporated by reference.

### TECHNICAL FIELD

The present subject matter described herein generally relates to a device and a method for tracking, and more particularly a device and a method for controlling operations of a tracking device installed in a surgical kit.

### BACKGROUND

Generally a tracking system is used for observation of objects on in transition and supplying a timely ordered sequence of respective location data to a user. Particularly the tracking systems are used in distribution and logistics of numerous products. In other words, tracking and tracing may be understood as a process of determining the current and past locations and other information of a unique item or property.

Currently, there are numerous tracking systems available in the market, for example a bar code, a gate, a Global Positioning Systems, bar-code systems, an automatic identification (RFID auto-id). But such conventional tracking systems fail when utilized in the healthcare domain. Generally, hospitals and surgical kit providers utilized conventional tracking system in order to track a variety of assets, including everything from wheelchairs to IV pumps. But conventional tracking systems implemented on surgical tools fail, because the surgical tools are regularly put through harsh sterilization process(es) involving intense heat and significant moisture. Further, the conventional tracking systems consume a substantial amount of battery for tracking resulting in frequent battery change.

### SUMMARY

Disclosed are devices and methods for enabling controlling operations of a tracking device installed in a surgical kit, and it is to be understood that this application is not limited to the particular device, systems, and methodologies described, as there can be multiple possible embodiments which are not expressly illustrated in the present disclosures. It is also to be understood that the terminology used in the description is for the purpose of describing the particular implementations or versions or embodiments only, and is not intended to limit the scope of the present application. This summary is provided to introduce aspects related to a device and a method for controlling operations of a tracking device installed in a surgical kit.

In one implementation, a device for controlling operations of a tracking device installed in a surgical kit is disclosed. In one aspect, the device includes a memory and a machine control unit coupled to the memory. Further, the machine control unit may be capable of executing instructions in the memory to perform one or more steps. In the aspect, the machine control unit may receive one or more signals from an accelerometer. The accelerometer may be configured to transmit the one or more signals upon detecting change in a state of the surgical kit. Further, the state may be one of a stationary state and an in-motion state. Upon receiving, the machine control unit may activate the tracking device based on the one or more signals. Further to activating, the machine control unit may obtain a location co-ordinate of the surgical kit from the tracking system, wherein the tracking device includes a GPS module. Subsequent to obtaining, the machine control unit may transmit the state of the surgical kit and the location co-ordinate of the surgical kit via a communication channel based on a signal strength of the communication channel. Upon transmitting, the machine control unit may deactivate the tracking device, thereby controlling operations of a tracking device installed in a surgical kit.

In one implementation, a method for controlling operations of a tracking device installed in a surgical kit is disclosed. In one aspect, the method may include receiving one or more signals from an accelerometer. The accelerometer may be configured to transmit the one or more signals upon detecting change in a state of the surgical kit. Further, the state may be one of a stationary state and an in-motion state. Further, the method may include activating the tracking device based on the one or more signals. Furthermore, the method may include obtaining a location co-ordinate of the surgical kit from the tracking system, wherein the tracking device includes a GPS module. The method may include transmitting the state of the surgical kit and the location co-ordinate of the surgical kit via a communication channel based on a signal strength of the communication channel. The method may also include deactivating the tracking device, thereby controlling operations of a tracking device installed in a surgical kit.

In one implementation, a device for controlling operations of a tracking device installed in a surgical kit is disclosed. In one aspect, the device may include an accelerometer. Further the accelerometer may transmits one or more signals upon detecting change in state of the surgical kit, wherein the state is one of a stationary state and an in-motion state. The device may further include a temperature sensor. The temperature sensor may transmits a first signal upon detecting one of a temperature rise above a first threshold during sterilization of the surgical kit and a second signal upon detecting a temperature drop below a second threshold upon completion of the sterilization of the surgical kit. The device may furthermore include a machine control unit electronically coupled to the accelerometer and the temperature sensor. The machine control unit may be further configured to receive one or more signals from the accelerometer and the temperature sensor, and perform one of activation and a deactivation of the tracking system based on the one or more signals and predefined conditions. The machine control unit may be furthermore configured to transmit one or more of the state of the surgical kit, a location co-ordinate of the surgical kit and a sterilization completion status via a communication channel based on the strength of the communication channel.

### BRIEF DESCRIPTION OF THE DRAWINGS

The foregoing detailed description of embodiments is better understood when read in conjunction with the appended drawings. For the purpose of illustrating of the present subject matter, an example of construction of the present subject matter is provided as figures; however, the invention is not limited to the specific method and system disclosed in the document and the figures.

The present subject matter is described detail with reference to the accompanying figures. In the figures, the left-most digit(s) of a reference number identifies the figure in which the reference number first appears. The same numbers are used throughout the drawings to refer various features of the present subject matter.

FIG. 1 illustrates a network implementation of a device for controlling operations of a tracking device installed in a surgical kit, in accordance with an embodiment of the present subject matter.

FIG. 2 illustrates a circuit diagram of the device for controlling operations of a tracking device installed in a surgical kit, in accordance with an embodiment of the present subject matter.

FIG. 3 illustrates the machine control unit and its subcomponents for controlling operations of a tracking device installed in a surgical kit, in accordance with an embodiment of the present subject matter.

FIG. 4 illustrates a method for controlling operations of a tracking device installed in a surgical kit, in accordance with an embodiment of the present subject matter.

### DETAILED DESCRIPTION

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Although any a device and a method for enabling controlling operations of a tracking device installed in a surgical kit, similar or equivalent to those described herein can be used in the practice or testing of embodiments of the present disclosure, the exemplary, a device and a method for enabling controlling operations of a tracking device installed in a surgical kit are now described. The disclosed embodiments for enabling a user to remotely perform an action in a real world via a virtual reality environment are merely examples of the disclosure, which may be embodied in various forms.

Various modifications to the embodiment will be readily apparent to those skilled in the art and the generic principles herein may be applied to other embodiments for controlling operations of a tracking device installed in a surgical kit. However, one of ordinary skill in the art will readily recognize that the present disclosure for controlling operations of a tracking device installed in a surgical kit is not intended to be limited to the embodiments described, but is to be accorded the widest scope consistent with the principles and features described herein.

In one implementation, a device and method for controlling operations of a tracking device installed in a surgical kit, is described. In the embodiment the device may comprise at least an accelerometer, a temperature sensor, and a machine control unit electronically coupled to the accelerometer and the temperature sensor.

In the embodiment, the accelerometer may be configured transmit one or more signals upon detecting change in state of the surgical kit. In one example, the state of surgical kit may be one of a stationary state and an in-motion state. In one other example, the accelerometer may be configured transmit one or more signals after a predefined time interval, such as five min, upon detecting change in state of the surgical kit. In the example the change in state may be from an “in-motion” state to a “stationary” state or from the “stationary” state to the “in-motion state”.

Further in the embodiment, the temperature sensor may be configured transmits a first signal upon detecting one of a temperature rise above a first threshold during sterilization of the surgical kit and a second signal upon detecting a temperature drop below a second threshold upon completion of the sterilization of the surgical kit. In one example, the temperature sensor may transmit a first signal when the temperature of the surgical kit rises above 60 degree and transmit a second signal when the temperature of the surgical kit falls below 50 degrees.

Furthermore in the embodiment, the machine control unit may be configured to receive one or more signals from the accelerometer and the temperature sensor. Upon receiving the one or more signal, the machine control unit may be configured to perform one of activation and a deactivation of the tracking system based on the one or more signals. Further to performing, the machine control unit may be configured to transmit one or more of the state of the surgical kit, a location co-ordinate of the surgical kit and a sterilization completion status via a communication channel, upon activation or deactivation. In an example, battery health information, identification information to server and additional information can also be added, if required, to the transmission. In one example, the transmission based on the strength of the communication channel, thereby controlling operations of the tracking device installed in the surgical kit. In the example, if the strength of the communication channel is below a predefined threshold, the state of the surgical kit, the location co-ordinate of the surgical kit and the sterilization completion status is stored. Subsequently when the signal strength of the communication channel exceeds the predefined threshold the state of the surgical kit, the location co-ordinate of the surgical kit and the sterilization completion may be transmitted via the communication channel when.

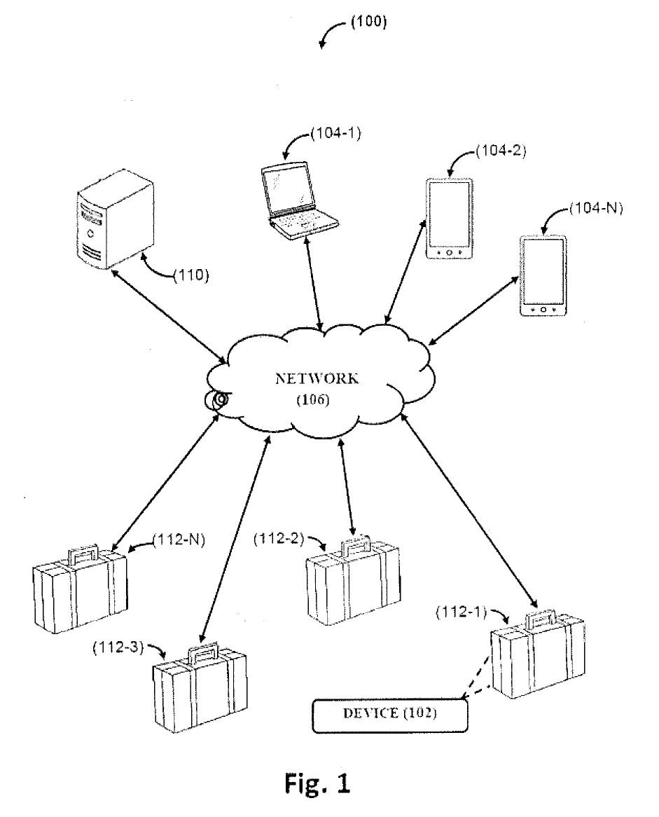

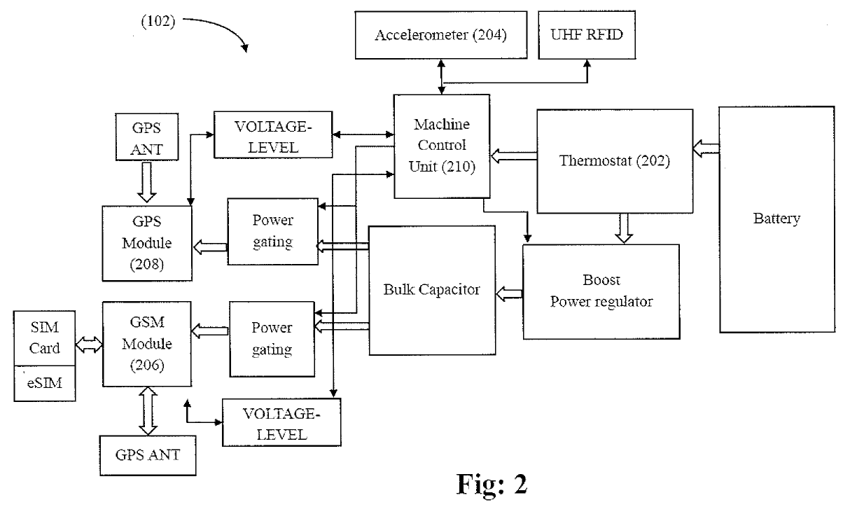

Referring now to FIG. 1, a network implementation 100 of a device 102 for controlling operations of a tracking device installed in a surgical kit 112-1, 112-2, 112-3,112-N, in accordance with an embodiment of the present subject matter may be described. Referring now to FIG. 2, a circuit diagram of the device 102 for controlling operations of a tracking device installed in a surgical kit 112-1, 112-2, 112-3, 112-N, in accordance with an embodiment of the present subject matter may be described.

In one embodiment, the present subject matter is explained considering that the device(s) 102 implemented on the surgical kits 112-1, 112-2, 112-3, 112-N, herein after jointly and individually referred to as the surgical kit 112. In one example the device 102 may be implemented as a device 102 connected to the network 106. It may also be understood that the device 102 supports a plurality of browsers and all viewports. Examples of the plurality of browsers may include, but not limited to, Chrome™, Mozilla™, Internet Explorer™, Safari™, and Opera™. It will also be understood that the device 102 may be accessed by multiple users through one or more user devices 104. In one example, the user device 104 may be a laptop 104-1, a mobile, a smartphone 104-2, desk top computer and the like 104-N. Furthermore, the device 102 may be communicatively coupled to one or more database for storing data and one or more servers 110 for transmitting data. In one example, the database may be any of the relationship database and the like. Further the device installed on the surgical kit 112 may be communicatively coupled to the one or more user devices 104, the database, the server 110 via communication channel, herein after referred to as a network 106

In one implementation, the network 106 may be a wireless network, a wired network or a combination thereof. The network 106 can be implemented as one of the different types of networks, such as intranet, local area network (LAN), Wireless Personal Area Network (WPAN), Wireless Local Area Network (WLAN), wide area network (WAN), the internet, and the like. The network 106 may either be a dedicated network or a shared network. The shared network represents an association of the different types of networks that use a variety of protocols, for example, MQ Telemetry Transport (MQTT), Extensible Messaging and Presence Protocol (XMPP), Hypertext Transfer Protocol (HTTP), Transmission Control Protocol/Internet Protocol (TCP/IP), Wireless Application Protocol (WAP), and the like, to communicate with one another. Further the network 106 may include a variety of network devices, including routers, bridges, servers, computing devices, storage devices, and the like.

In the embodiment, the device 102 for controlling operations of a tracking device installed in a surgical kit 112-1, 112-2, 112-3, 112-N, in accordance with an embodiment of the present subject matter may be described. In the embodiment, the device 102 comprises an accelerometer 204, a temperature sensor 202, a machine control unit 210, and a tracking system 208. In one example, the device 102 may comprises other components. The table 1 illustrates few other components that may be utilized in the device 102.

TABLE 1
An exemplary list of component the device 102 comprises.

| Part | Description |
|---|---|
| Battery | Lithium Thionyl Chloride (Li-SOCI2) |
| | Battery, Capacity: 8500 mAh |
| Battery with Super | Battery with integrated Super capacitor |
| capacitor module | |
| Thermostat | IC THERMOSTAT PRESET SOT23-5 |
| GPS Module | Jupiter SE868 AS GPS module w/ Antenna |
| GPS Module | GPS module |
| 4.7K ohms | RES SMD 4.7K OHM 1% 1/10 W 0603 |
| 47K ohms | RES SMD 47K OHM 1% 1/10 W 0603 |
| 1K ohms | RES SMD 1K OHM 1% 1/10 W 0603 |
| 100K ohms | RES SMD 100K OHM 1% 1/10 W 0603 |
| 3.3K ohms | RES SMD 3.3K OHM 1% 1/10 W 0603 |
| NPN Transistor | TRANS NPN 65 V 0.1A SOT-23 |
| LED | LED GREEN CLEAR 0805 SMD |
| GSM Module | QUAD-BAND GSM/GPRS |
| GSM Module | GSM/3G module |
| RTC Battery | CAP 80 MF 3.3 V SURFACE MOUNT |
| RF Antenna | ONBOARD SMD 868/915 |
| BT Antenna | IC ANTENNA ONBOARD 2400 SMD |
| ESD Diode | TVS Diode Arrays 150 W 6.1 V Quad Array |
| SIM Card holder | CONN MICRO SIM CARD PUSH-PULL |
| NPN Transistor | TRANS NPN 65 V 0.1 A SOT-23 |
| 100K ohms | RES SMD 100K OHM 1% 1/10 W 0603 |
| 3.3K ohms | RES SMD 3.3K OHM 1% 1/10 W 0603 |
| 3.3K ohms | RES SMD 3.3K OHM 1% 1/10 W 0603 |
| LED | LED GREEN CLEAR 0805 SMD |
| DC-DC Converter | Ultra-Low Quiescent Current |
| | Low-Dropout Linear Regulator |
| Buck boost regulator | IC REG BCK BST PROG 50 MA 20QFN |
| Super Capacitor | Supercapacitors/Ultra capacitors RADIAL |
| | 2.7 V 50 F. |
| Accelerometer | ACCEL 2-16G I2C/SPI 16LGA |
| High Side Switch | IC LOAD SW HISIDE 3A 4-MLF |
| High Side Switch | IC LOAD SW HGH SIDE 1.2 A SC70-6 |
| Load Switch | Low on Resistance Load Switch With |
| | Controlled Turn-on |
| Microcontroller | MIXED SIGNAL MICROCONTROLLER |
| EEPROM | IC EEPROM 1 MBIT 1 MHZ 8SO |

In the embodiment, the accelerometer 204 may be configured transmit one or more signals upon detecting change in state of the surgical kit. In one example, the state of surgical kit may be one of a stationary state and an in-motion state. In one other example, the accelerometer 204 may be configured transmit one or more signals after a predefined time interval, such as five min, upon detecting change in state of the surgical kit. In the example the change in state may be from in-motion state to stationary state or from stationary state to in-motion state.

Further in the embodiment, the temperature sensor 202, alternatively also known as thermostat 202, may be configured transmits a first signal upon detecting one of a temperature rise above a first threshold during sterilization of the surgical kit 112-1, 112-2, 112-3, 112-N and a second signal upon detecting a temperature drop below a second threshold upon completion of the sterilization of the surgical kit 112-1, 112-2, 112-3, 112-N. In one example, the temperature sensor 202 may transmit a first signal when the temperature of the surgical kit 112-1, 112-2, 112-3, 112-N rises above 60 degree and transmit a second signal when the temperature of the surgical kit 112-1, 112-2, 112-3, 112-N falls below 50 degrees.

Furthermore in the embodiment, the machine control unit 210 may be configured to receive one or more signals from the accelerometer 204 and the temperature sensor 202. Upon receiving the one or more signal, the machine control unit 210 may be configured to perform one of an activation and a deactivation of the tracking system based on the one or more signals and a predefined conditions. Further to performing, the machine control unit 210 may be configured to transmit one or more of the state of the surgical kit 112-1, 112-2, 112-3, 112-N, a location co-ordinate of the surgical kit 112-1, 112-2, 112-3, 112-N and a sterilization completion status via a communication channel 106. In an example, battery health information, identification information to server and additional information can also be added, if required, to the transmission. In one example, the transmission may be based on the strength of the communication channel 106, thereby controlling operations of the tracking device installed in the surgical kit 112-1, 112-2, 112-3, 112-N. In the example, if the strength of the communication channel 106 is below a predefined threshold, the state of the surgical kit 112-1, 112-2, 112-3, 112-N, the location co-ordinate of the surgical kit 112-1, 112-2, 112-3, 112-N and the sterilization completion status is stored. Subsequently when the signal strength of the communication channel 106 exceeds the predefined threshold the state of the surgical kit 112-1, 112-2, 112-3, 112-N, the location co-ordinate of the surgical kit 112-1, 112-2, 112-3, 112-N and the sterilization completion may be transmitted via the communication channel when.

Further the state of the surgical kit 112-1, 112-2, 112-3, 112-N, the location coordinate of the surgical kit 112-1, 112-2, 112-3, 112-N and the sterilization completion may be received by a server 110 based on the transmission. In one example, the server 110 may collate and store the all the data related to plurality of surgical units. Furthermore, the server may provide various updates to the user device 104.

Furthermore in the embodiment, the device 102 may also comprise an airplane detection algorithm configured to detect if the surgical kit 112-1, 112-2, 112-3, 112-N is inside an airplane using Accelerometer, GPS data and a predefined assessment criterion. Further the device 102 may switch off the all the electronics and disconnect the battery based if the detection is positive, thus confirming the surgical kit is inside an airplane.

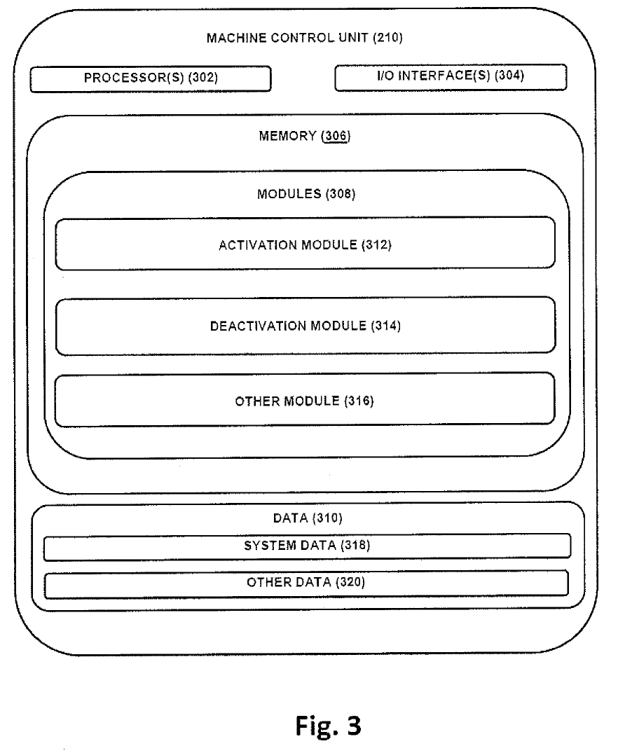

Referring now to FIG. 3, the machine control unit 210 is illustrated in accordance with an embodiment of the present subject matter. In one embodiment, the machine control unit 210 may include at least one processor 302, an input/output (I/O) interface 304, and a memory 306. The at least one processor 302 may be implemented as one or more microprocessors, microcomputers, microcontrollers, digital signal processors, central processing units, state machines, logic circuitries, and/or any devices that manipulate signals based on operational instructions. Among other capabilities, the at least one processor 202 may be configured to fetch and execute computer-readable instructions stored in the memory 206.

The I/O interface 304 may include a variety of software and hardware interfaces, for example, a web interface, a graphical user interface, and the like. The I/O interface 304 may allow the machine control unit 210 to interact with the user directly or through the user devices 104. Further, the I/O interface 304 may enable the machine control unit 210 to communicate with other computing devices, such as web servers and external data servers (not shown). The I/O interface 304 can facilitate multiple communications within a wide variety of networks and protocol types, including wired networks, for example, LAN, cable, etc., and wireless networks, such as WLAN, cellular, or satellite. The I/O interface 304 may include one or more ports for connecting a number of devices to one another or to another server.

The memory 306 may include any computer-readable medium or computer program product known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes. The memory 306 may include modules 308 and data 310.

The modules 308 include routines, programs, objects, components, data structures, etc., which perform particular tasks or implement particular abstract data types. In one implementation, the modules 308 may include an activation module 312, a deactivation module 314 and other module 218. The other modules 318 may include programs or coded instructions that supplement applications and functions of the machine control unit 210. The modules 308 described herein may be implemented as software modules that may be executed in the cloud-based computing environment of the machine control unit 210.

The memory 306, amongst other things, serves as a repository for storing data processed, received, and generated by one or more of the modules 308. The memory 306 may include data generated as a result of the execution of one or more modules in the other module 320. In one implementation, the memory may include data 310. Further, the data 310 may include a system data 320 for storing data processed, computed received and generated by one or more of the modules 308. Furthermore, the data 310 may include other data 322 for storing data generated as a result of the execution of one or more modules in the other module 318.

In one implementation, at first, a user may use the user device 104 to access the device 102 and in turn the machine control unit 210. The user may register using the I/O interface 204 in order to use the device 102. In one aspect, the user may access the I/O interface 204 of the device 102 for obtaining information or providing input information. In one other embodiment, the user may user the user device 104 to register at a central server in order to obtain or receive data from or about the device 102 and the surgical kit. In one implementation the device 102 utilizing the machine control unit 210 may automatically provide information to the user through I/O interface 304, and user device 104. In one embodiment, the machine control unit 210 may comprises an activation module 312 and deactivation module 314 for performing one or more instructions.

In the embodiment, the activation module 312 may receive one or more signals from an accelerometer. In one example, the accelerometer may be configured to transmit the one or more signals upon detecting change in a state of the surgical kit. In one example, transmission of the one or more signal may be immediately or after a predefined time delay upon detecting the change in the state of the surgical kit. In the example, the state may be one of a stationary state and an in-motion state. In one implementation of the example, the accelerometer may transmit a signal when the surgical kit initiates motion. Further, the accelerometer may be configured to transmit the signal when the speed of motion of the surgical exceeds a predefined speed. In one implementation of the example, the accelerometer may transmit a signal when the surgical kit stops motion. Further, the accelerometer may be configured to transmit the signal when the speed of motion of the surgical is zero for a predefined amount of time. Further, the activation module 312 may store the signal in the system data 318.

In the embodiment, the activation module 312 may activate the tracking device based on the one or more signals. Upon activating the tracking device, the deactivation module 314 may obtain a location co-ordinate of the surgical kit from the tracking device. Further the tracking device may comprise a GPS module 208. Further to obtain the location co-ordinate of the surgical kit the deactivating module 314 may utilize the communication channel such as GSM module 206 for transmitting the state of the surgical kit and the location co-ordinate of the surgical kit. Further the transmission may be based on signal strength of the communication channel. In one example, if the signal strength is above a predefined threshold the deactivating module 314 may transmit the state of the surgical kit and the location co-ordinate of the surgical kit immediately. In one example, if the signal strength is below a predefined threshold the deactivating module 314 may store the state of the surgical kit and the location co-ordinate of the surgical kit and transmit he surgical kit and the location co-ordinate of the surgical kit when the signal strength is above a predefined threshold. Upon transmitting or storing, the deactivating module 314 may deactivate the tracking device, thereby controlling operations of a tracking device installed in a surgical kit.

In one more embodiment, a deactivation module 314 may receive a first signal from a temperature sensor. The temperature sensor 202 may be configured to transmit the first signal upon detecting a temperature rise above a first threshold during sterilization of the surgical kit. For example, the temperature sensor 202 may transmit a first signal when the temperature of the surgical kit exceeds 60 degrees. The temperature rises is indicative of initiation of the sterilization of the surgical kit. Further to obtain the first signal, the deactivation module 314 may deactivate the tracking device based on the first signal.

In the one more embodiments, the activation module 312 may receive a second signal from the temperature sensor 202. The temperature sensor 202 may be configured to transmit the second signal upon detecting a temperature drop below a second threshold. The temperature drop represents completing the sterilization of the surgical kit. For example, the temperature sensor 202 may transmit a second signal when the temperature of the surgical kit decrees below 50 degrees. Upon receiving the second signal, the activation module 312 may activate the tracking device based on the second signal. Further to activating the activation module 312 transmit a sterilization completion status via the communication channel such as GSM module 206. In an example, battery health information, identification information to server and additional information can also be added, if required, to the transmission. Further the transmission may be based on signal strength of the communication channel. In one example, if the signal strength is above a predefined threshold, the activation module 314 may transmit the sterilization completion status immediately. In one example, if the signal strength is below a predefined threshold the deactivating module 314 may store sterilization completion status along with the time stamp and transmit sterilization completion status along with the time stamp when the signal strength is above a predefined threshold. Upon transmitting or storing, the deactivating module 314 may deactivate the tracking device, thereby controlling operations of a tracking device installed in a surgical kit.

Exemplary embodiments for controlling operations of a tracking device installed in a surgical kit discussed above may provide certain advantages. Though not required to practice aspects of the disclosure, these advantages may include those provided by the following features.

Some embodiments of the device and the method enable power management of the tracking device.

Some embodiments of the device and the method enable battery optimizing.

Some embodiments of the device and the method protection of the tracking device during sterilization process.

Some embodiments of the device and the method enable automated inventory management.

Some embodiments of the device and the method enable low power design.

Some embodiments of the device and the method enable a battery for example, 4 AA high temperature Non rechargeable Lithium batteries to work for a year with update/refresh rate of 2 per day.

Some embodiments of the device and the method withstand temperature up to 125 degree Celsius in storage condition.

Some embodiments of the device and the method withstand autoclave temperature/pressure conditions due to a sealed mechanical enclosure design.

Some embodiments of the device and the method enable auto shutdown of electronics at high temperature.

Some embodiments of the device and the method comprise provision to configure modules with UHF RFID.

Some embodiments of the device and the method support's 2G/3G network for Global Band coverage.

Some embodiments of the device and the method support normal SIM or eSIM based on hardware design.

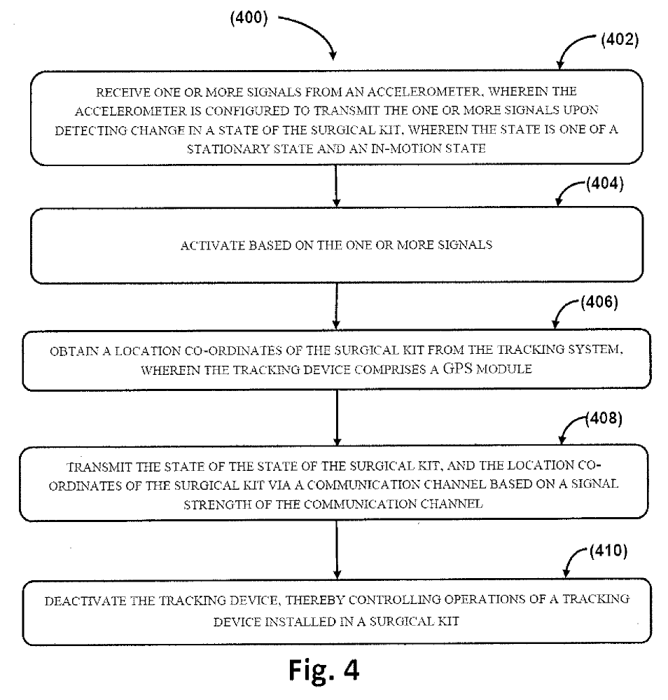

Referring now to FIG. 4, a method 400 for controlling operations of a tracking device installed in a surgical kit is shown, in accordance with an embodiment of the present subject matter. The method 400 may be described in the general context of computer executable instructions. Generally, computer executable instructions can include routines, programs, objects, components, data structures, procedures, modules, functions, etc., that perform particular functions or implement particular abstract data types.

The order in which the method 400 for controlling operations of a tracking device installed in a surgical kit is not intended to be construed as a limitation, and any number of the described method blocks can be combined in any order to implement the method 400 or alternate methods. Additionally, individual blocks may be deleted from the method 400 without departing from the spirit and scope of the subject matter described herein. Furthermore, the method can be implemented in any suitable hardware, software, firmware, or combination thereof. However, for ease of explanation, in the embodiments described below, the method 400 may be considered to be implemented in the above described device 102.

At block 402, one or more signals may be received from an accelerometer. Further, the accelerometer may be configured to transmit the one or more signals upon detecting change in a state of the surgical kit. Furthermore, the state is one of a stationary state and an in-motion state. In an implementation the activation module 312 may receive one or more signals an accelerometer. Further the receiving module 312 may store the one or more signals in the system data 320.

At block 404, the tracking device is activated based on the one or more signals. In an implementation, the activation module 312 may activate the tracking device.

At block 406, a location co-ordinate of the surgical kit may be obtained from the tracking system. Further, the tracking device may comprise a GPS module. In the implementation, the deactivation module 314 may obtain a location co-ordinate of the surgical kit and store the location co-ordinate of the surgical kit in the system data 320.

At block 408, the state of the surgical kit and the location co-ordinate of the surgical kit may be transmitted via a communication channel based on the signal strength of the communication channel. In the implementation, the deactivation module 314 may transmit the state of the surgical kit and the location co-ordinate of the surgical kit. Further, the deactivation module 314 may store the state of the surgical kit and the location co-ordinate of the surgical kit in system data 320.

At block 410, the tracking device is deactivated, thereby controlling operations of a tracking device installed in a surgical kit. In the implementation, the deactivation module 314 may deactivate the tracking device once the transmission is complete.

Exemplary embodiments discussed above may provide certain advantages. Though not required to practice aspects of the disclosure, these advantages may include a method and device for controlling operations of a tracking device installed in a surgical kit. Although implementations for methods and device for controlling operations of a tracking device installed in a surgical kit have been described in language specific to structural features and/or methods, it is to be understood that the appended claims are not necessarily limited to the specific features or methods described. Rather, the specific features, devices, systems and methods are disclosed as examples of implementations for controlling operations of a tracking device installed in a surgical kit.

We claim:

1. A device for controlling operations of a tracking device installed in a surgical kit, the device comprising:
a memory; and
a machine control unit coupled to the memory, wherein the machine control unit is capable of executing instructions to perform steps of:
receiving one or more signals from an accelerometer, wherein the accelerometer is configured to transmit the one or more signals upon detecting change in a state of the surgical kit, wherein the state is one of a stationary state and an in-motion state;
activating the tracking device based on the one or more signals;
obtaining a location co-ordinate of the surgical kit from the tracking system, wherein the tracking device comprises a GPS module;
transmitting the state of the surgical kit and the location co-ordinate of the surgical kit via a communication channel based on a signal strength of the communication channel; and
deactivating the tracking device, thereby controlling operations of the tracking device installed in the surgical kit.

2. The device of claim 1, the device further comprises
receiving a first signal from a temperature sensor, wherein the temperature sensor is configured to transmit the first signal upon detecting a temperature rise above a first threshold during sterilization of the surgical, wherein the temperature rises is indicative of initiation of the sterilization of the surgical kit;
deactivating the tracking device based on the first signal;
receiving a second signal from the temperature sensor, wherein the temperature sensor is configured to transmit the second signal upon detecting a temperature drop below a second threshold upon completion of the sterilization of the surgical kit, wherein the temperature drop is indicative of completion of the sterilization of the surgical kit;
activating the tracking device based on the second signal; and
transmitting a sterilization completion status, based on the activation, via the communication channel based on the signal strength of the communication channel.

3. The device of claim 1, the device further comprises
storing the state of the surgical kit, the location co-ordinate of the surgical kit, the sterilization completion status, and a time stamp when the signal strength of the communication channel is below a predefined threshold; and
transmitting the state of the surgical kit, the location co-ordinate of the surgical kit, the sterilization completion status and the time stamp via the communication channel when the signal strength of the communication channel exceeds the predefined threshold.

4. A method for controlling operations of a tracking device installed in a surgical kit, the method comprising:
receiving, by a machine control unit, one or more signals from an accelerometer, wherein the accelerometer is configured to transmit the one or more signals upon detecting change in a state of the surgical kit, wherein the state of the surgical kit is one of a stationary state and an in-motion state;
activating, by the machine control unit, the tracking device based on the one or more signals;
obtaining, by the machine control unit, a location co-ordinate of the surgical kit from the tracking system, wherein the tracking device comprises a GPS module;
transmitting, by the machine control unit, the state of the surgical kit, and the location co-ordinate of the surgical kit, via a communication channel, based on a signal strength of the communication channel; and
deactivating, by the machine control unit, the tracking device based on the transmission, thereby controlling operations of the tracking device installed in the surgical kit.

5. The method of claim 4, the method further comprises
receiving, by the machine control unit, a first signal from a temperature sensor, wherein the temperature sensor is configured to transmit the first signal upon detecting a temperature rise above a first threshold during sterilization of the surgical kit, wherein the temperature rises is indicative of initiation of the sterilization of the surgical kit;
deactivating, by the machine control unit, the tracking device based on the first signal;
receiving, by the machine control unit, a second signal from the temperature sensor, wherein the temperature sensor is configured to transmit the second signal upon detecting a temperature drop, below a second threshold upon completion of the sterilization of the surgical kit, wherein the temperature drop is indicative of completion of the sterilization of the surgical kit;
activating, by the machine control unit, the tracking device based on the second signal; and
transmitting, by the machine control unit, a sterilization completion status, based on the activation, via the communication channel based on the signal strength of the communication channel.

6. The method of claim 4, the method further comprises
storing, by the machine control unit, the state of the surgical kit, the location coordinate of the surgical kit, the sterilization completion and a time stamp, when the signal strength of the communication channel is below a predefined threshold; and
transmitting, by the machine control unit, the state of the surgical kit, the location co-ordinate of the surgical kit, the sterilization completion and the time stamp, via the communication channel, when the signal strength of the communication channel exceeds the predefined threshold.

7. A device for controlling operations of a tracking device installed in a surgical kit, the device comprising:
an accelerometer, wherein the accelerometer transmits one or more signals upon detecting change in a state of the surgical kit, wherein the state is one of a stationary state and an in-motion state
a temperature sensor, wherein the temperature sensor transmits a first signal upon detecting one of a temperature rise above a first threshold during sterilization of the surgical kit and a second signal upon detecting a temperature drop below a second threshold upon completion of the sterilization of the surgical kit, wherein the temperature rises is indicative of initiation of the sterilization of the surgical kit, and wherein the temperature drop is indicative of completion of the sterilization of the surgical kit; and
a machine control unit electronically coupled to the accelerometer and the temperature sensor, wherein the machine control unit is configured to:
receive one or more signals from the accelerometer and the temperature sensor;
perform one of an activation and a deactivation of the tracking system based on the one or more signals and a predefined conditions; and
transmit one or more of the state of the surgical kit, a location co-ordinate of the surgical kit and a sterilization completion status via a communication channel based on the strength of the communication channel, thereby controlling operations of the tracking device installed in the surgical kit.

8. The device of claim 7, wherein the machine control unit is further configured to
store the state of the surgical kit, the location co-ordinate of the surgical kit, the sterilization completion, and a time stamp when the signal strength of the communication channel is below a predefined threshold; and
transmit the state of the surgical kit, the location co-ordinate of the surgical kit, the sterilization completion, and the time stamp via the communication channel when the signal strength of the communication channel exceeds the predefined threshold.
